(19) United States
(12) Patent Application Publication
(10) Pub. No.: US 2005/0019421 A1
(43) Pub. Date: Jan. 27, 2005

(54) Disinfecting compositions and methods of making and using same

(75) Inventors: Terry R. Hobbs, St. Paul, MN (US); Jeffrey F. Andrews, Stillwater, MN (US); Sophia M. Czechowicz, Woodbury, MN (US); Luke E. Schallinger, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/625,271

(22) Filed: Jul. 23, 2003

(51) Int. Cl. A61K 33/40; A61K 31/19

(52) U.S. Cl. 424/616; 514/568

(57) ABSTRACT

The present invention provides a composition, comprising: greater than about 0.1% by weight hydrogen peroxide; an aromatic acid component; surfactant; optionally, a solvent; and a carrier. The composition of the invention is useful as a disinfecting composition for killing microorganisms such as bacterium (including Mycobacterium), spores and fungi. The composition provides a pathogenic bacteria kill rate of 99.9% in about 30 seconds when bacteria are exposed to the composition and is effective in providing a Mycobacterium kill of 10⁶ with two minutes or less. Moreover, the compositions of the invention are generally more resistant to catalase deactivation than, for example, an aqueous solution of hydrogen peroxide. The concentration of hydrogen peroxide within the composition may range from about 1% by weight to about 7% by weight and the concentration of aromatic acid component may range from about 0.1% by weight to about 5% by weight. The invention also provides a method for disinfection of a substrate utilizing the composition. The composition of the invention may be used in the foregoing method on a medical instrument, such as an endoscope or the like. Applying the compositions to a substrate may be accomplished in any of a variety of application methods such as by roll coating, dipping, spraying, or rotational tumbling. The composition may be applied to the substrate for a period of time ranging from about 30 seconds to about ten minutes. In this aspect, the invention can further comprise drying the substrate after removing the composition.

The present invention relates to a composition that is useful in high level disinfection, methods of making the composition and methods of use.

### BACKGROUND OF THE INVENTION

A disinfecting agent is a composition that, when applied to a surface or the like will kill a wide spectrum of microorganisms such as bacteria, fungi and viruses. The term “high level disinfectant” (“HLD”) designates a class of disinfecting agents that can kill 10⁶ mycobacteria and have the ability to kill bacterial endospores, the most difficult of all microorganisms to kill. A high level disinfectant can reduce spore populations and at the same time destroy less hardy pathogens such as mycobacteria, fungi, bacteria, and viruses. A “sterilant” is an agent capable of killing 10⁶ bacterial endospores.

Cleaning, disinfection and/or sterilization of medical instruments and devices is a common practice in the health care industry, and improvements to this practice are desired for the further enhancement of hygiene and patient safety. Effective disinfection must act against a wide spectrum of microorganisms including those resistant to common antibacterial agents while not damaging the instruments and devices upon which they are used and without posing significant health or environmental issues of their own.

Chemical compounds capable of disinfecting or sterilizing an instrument or a surface thereof include peroxy compounds, hydrogen peroxide, chlorine compounds, aldehydes, and phenolics. These compounds and the compositions containing them have been used for disinfecting surfaces and heat sensitive medical devices such as endoscopes, for example. Mycobacteria are generally much more difficult to kill in comparison to fungi, other bacteria, and viruses. Microorganisms from the Mycobacterium genus have been identified by the United States Food and Drug Agency (“FDA”) as the key organism to be used in establishing the disinfection time of a high level disinfectant. Tuberculosis, caused by Mycobacterium tuberculosis, is a key pathogenic organism of concern especially with the rise of antibiotic resistant strains. Approved non-pathogenic surrogates include Mycobacterium terrae and Mycobacterium bovis.

Although several commercial products are now available for high level disinfection, these products are often slow in achieving a desired level of disinfection and may suffer from one or more other disadvantages. For example, glutaraldehyde has been used in high level disinfection at a 2% level in an aqueous solution. But, disinfection times are typically as long as 20 to 45 minutes. Although the disinfection times can be reduced with heating (e.g., to 35° C.), material compatibility and health issues have complicated the safety and efficacy picture for this compound. Likewise, peracetic acid and orthophthaldehyde have also been used in high level disinfection, often with undesired disinfection times and/or with undesired material compatibility and concentration related health or safety issues.

Hydrogen peroxide is known to possess broad germicidal properties with an ability to kill organisms through oxidative action. At lower concentrations (e.g., <6%), it is safe to handle and is considered environmentally friendly because it readily decomposes into oxygen and water. One disadvantage in the use of hydrogen peroxide is its rate of disinfection or its kill rate can be rather slow, even when used to eliminate common bacteria such as Staphyloccocus aureus. Although the disinfection rate or kill rate can be increased simply by increasing the hydrogen peroxide concentration, the increase in the kill rate is frequently obtained at the expense of safety because the more concentrated peroxide solutions are also strong oxidizing agents.

A water-soluble acid such as peracetic or acetic acid can be combined with hydrogen peroxide to improve its efficacy, and this approach has been used by some manufacturers of disinfectants who have added a second active such as phosphoric, peracetic, or a food grade acid. However, these combinations of peroxide and acid still require longer times to achieve high level disinfection (e.g., more than 5 minutes) and have generally been relatively poor performers. They also have poor materials compatibility because of their low pH (typically <3) and oxidizing ability. Reports of shorter disinfection times may utilize formulations that combine an acid such as succinic acid with a higher level of peroxide, (e.g., 13.4%), thus negating the attractive safety feature of a more dilute hydrogen peroxide solution. Hydrogen peroxide concentrations of 8% or more are classified by the United States Department of Transportation as strong oxidizers that require special shipping conditions.

There is a need for a disinfectant capable of high level disinfection and exhibiting an improved rate of high level disinfection. It is desirable to provide such a disinfectant in a safe and fast acting form capable of killing a broad range of microorganisms including mycobacteria, fungi, and bacteria while also having improved materials compatibility.

### SUMMARY OF THE INVENTION

The present invention provides a composition, comprising:
Greater than about 0.1% by weight hydrogen peroxide;
Aromatic acid component;
Surfactant;
Optionally, a solvent; and
A carrier.

As used herein, “aromatic acid” refers to a carboxylic acid functionality directly attached to a benzene ring.

The composition of the invention is useful as a disinfecting composition for killing microorganisms such as bacterium (including Mycobacterium), spores and fungi. The composition provides a pathogenic bacteria kill rate of 99.9% in about 30 seconds when bacteria are exposed to the composition and is effective in providing a mycobacteria kill of 10⁶ in two minutes or less. The compositions of the invention are generally much more resistant to catalase deactivation than, for example, an aqueous solution of hydrogen peroxide. In achieving a composition having these qualities, the aromatic acid, surfactant, and the hydrogen peroxide are present within the composition in synergistic amounts to provide improved kill rates of microorganisms (e.g., bacteria, fungi and viruses) when applied to a substrate. In addition to the foregoing components, the compositions of the invention may further comprise one or more acidulant such as phosphoric acid, sulfuric acid, caprylic acid, capric acid, lauric acid, or citric acid or combinations of the foregoing.

The aromatic acid component may be selected from benzoic acid, alkyl derivatives of benzoic acid, hydroxybenzoic acids, halogenated benzoic acids, phthalic acid, terephthalic acid, derivatives of hydroxybenzoic acids such as acetylsalicylic acid, napthoic acid and combinations of the foregoing. Suitable surfactants may be anionic or nonionic, and the solvent may be glycols, alcohols, aprotic amides, esters, polyethers or combinations of the foregoing. The compositions of the invention may further comprise one or more optional constituents such as corrosion inhibitors, antifoaming agents, foaming agents, pH adjusting agents, coloring agents, peroxide stabilizing agents, fragrances, and chelating agents.

In another aspect of the invention, the concentration of hydrogen peroxide may range from about 1% by weight to about 7% by weight and the concentration of aromatic acid component may range from about 0.1% by weight to about 5% by weight.

In still another aspect, the invention provides a method for disinfecting, the method comprising:

Applying the foregoing composition to a substrate;
Allowing the composition to remain in contact with the substrate for a period of time to kill microorganisms thereon; and
Removing the composition from the substrate.

The compositions of the invention may be used in the foregoing method on a medical instrument, such as an endoscope or the like. Applying the compositions to a substrate may be accomplished in any of a variety of application methods such as by roll coating, dipping, spraying, or rotational tumbling. The composition may be applied to the substrate for a period of time ranging from about 30 seconds to about ten minutes. In this aspect, the invention can further comprise drying the substrate after removing the composition.

In still another aspect, the invention provides a method for making the foregoing composition, the method comprising combining greater than about 0.1% by weight hydrogen peroxide; aromatic acid component; surfactant; an optional solvent; and a carrier such as water.

Those skilled in the art will further appreciate the foregoing aspects of the invention upon consideration of the remainder of the disclosure. It is also contemplated that equivalents to the described components and to the composition of the invention are possible but are as yet unforeseen. Nonetheless, such equivalents are within the scope of the invention.

### DETAILED DESCRIPTION OF PREFERRED EMBODIMENTS

The invention provides compositions capable of high level disinfection. The compositions of the invention comprise hydrogen peroxide, an aromatic acid component, a surfactant and a carrier. Because aromatic acids are substantially insoluble in water, an appropriate solvent and/or surfactant system is typically included to create a stable, aqueous based disinfectant. The compositions of the invention provide a rapid disinfection capability in the presence of organic soil and against organisms that produce catalase. Moreover, the compositions of the invention provide an improved rate of kill and an improved spectrum of activity against microorganisms while also exhibiting an improved efficacy against catalase producing organisms such as S. aureus.

The compositions of the invention are useful for disinfecting medical devices because they are easy to handle, generally compatible with other materials, and require a short contact time to achieve high level disinfection. The compositions of this invention superior to previously described disinfectant chemistries. Compared to known hydrogen peroxide formulations, the compositions of this invention provide a shorter time to achieve disinfection, improved efficacy, improved safety, and improved compatibility with other materials.

The compositions of the invention comprise hydrogen peroxide and will normally comprise greater than about 0.1% hydrogen peroxide and typically from 0.1 to 7.0 wt % hydrogen peroxide in a ready to use formulation. Typically, the amount of peroxide is from 1 to 6 wt %, and often will range between 3 and 5 wt %. The compositions of the invention may be provided in concentrated form wherein the peroxide concentration will typically range from about 5% to about 25%. The actual concentration of peroxide in a composition will depend on the particular application. Higher concentrations of peroxide may be desired if rapid disinfection is required while lower concentrations are typically used in compositions that may come in contact with human skin. For example, hydrogen peroxide at a weight percentage of 3% is a typical level in over the counter antiseptics. The low levels of hydrogen peroxide (typically 3 -5%) required for the ready to use compositions of the invention are sufficient to provide high level disinfection in a relatively short time.

While hydrogen peroxide is most commonly used in the inventive compositions described herein, other peroxides are also suitable such as perborates, percarbamates, percarbonates, and perorganic acids. Moreover, the compositions of the invention are typically aqueous based microemulsions, so that water insoluble peroxides can be used as well including perbenzoic acid or benzoyl peroxide.

Another component of the invention is an aromatic acid. The aromatic acid component may comprise one or more aromatic acids in combination, each aromatic acid may comprise one or more acid groups. Suitable organic acids for use in the compositions of the invention include but are not limited to: benzoic acid and its alkyl derivatives, hydroxybenzoic acids (e.g., salicylic acid), halogenated benzoic acids, phthalic acid, terephthalic acid, orthophthalic acid, acetylsalicylic acid, and napthoic acid. Typically, the aromatic acid is used in the composition in conjunction with one of its salts to provide buffering capacity to the composition. The compositions of the invention often comprise benzoic acid in conjunction with an alkali metal salt thereof such as sodium benzoate because these compounds have low toxicity and are widely used as food preservatives. Additionally, benzoic acid has a pKa of 4.2, making it a suitable buffer for the typical pH range of 3.5 to 5.0. Alternatively, the compositions of this invention may utilize a salt of an aromatic acid used in conjunction with one or more acidulants such as phosphoric acid, sulfuric acid, caprylic acid, capric acid, lauric acid, or citric acid. One such composition comprises sodium benzoate and phosphoric acid. As mentioned, the pH of the composition is typically in the range from about 3.5 to about 5.0 and can be controlled by altering the relative concentration of aromatic acid and salt or by altering the pH with acidulants or traditional bases such as sodium hydroxide or triethanolamine. Acidulants may be included in the compositions of the invention principally for pH control or adjustment as well as other uses that will be appreciated by those of ordinary skill in the art.

Salt of the aromatic acid in the compositions of the invention may be included in formulating the compositions. In either concentrated compositions or in ready to use compositions, the concentration of aromatic acid typically corresponds at least to the amount needed to provide a desired disinfecting action in the composition and typically includes at least the amount of salt needed to provide an identified synergy when combined in the composition with peroxide to perform in a desired manner as a disinfecting composition. Additionally, the salt and acid combination can function as a buffering agent for the composition. In the ready to use compositions of the invention, the concentration of the aromatic acid is normally in the range from 0.1 to 5.0 wt %, typically from 0.1 to 3.0% and often in the range from 0.6 to 1.5 wt %. Salts of the aromatic acid may be included within the ready to use compositions at a concentration typically less than 3.0% by weight and normally less than 1% by weight. In general, higher acid concentrations will be used in concentrated forms of the compositions wherein the aromatic acid concentration may range from about 1.0% to about 5.0%. Higher acid concentrations than those mentioned may be used in either the concentrate or in the ready to use formulations, but such concentrations generally have not been necessary for satisfactory antimicrobial activity and higher concentrations of acid tend to be more difficult to dissolve. Moreover, higher acid concentrations may be less desired from the perspective of also requiring higher solvent and/or surfactant concentrations. Generally, compositions intended solely to provide activity against bacteria and viruses can comprise lower concentrations of acid while compositions providing Mycobacterium activity will normally comprise higher concentrations of acid.

Benzoic acid and salicylic acid have both been found to be highly effective when included in the compositions of the invention in that they provide a synergistic effect when combined with peroxide. While hydrogen peroxide is known to provide mycobactericidal activity, it is typically very slow-acting in the aforementioned concentration range. Moreover, benzoic acid is not normally considered to be a mycobactericidal agent. However, the combination of benzoic acid and hydrogen peroxide, as described herein, provide a significantly increased bactericidal activity. The combination of the two have surprisingly fast kill rates (e.g., 2 minutes or less for a 10⁶ log reduction) against a broad spectrum of pathogens including mycobacteria and fungi. In addition, the combination can kill spores at longer exposure time. These features are not attainable when either component has been used alone or when an aliphatic acid has been used instead of the aromatic acid. In addition, benzoic acid and its salt, sodium benzoate, are effective when combined in a composition as acid/salt because they are both oxidatively stable. Typically the acid and salt are provided in the ready to use compositions of the invention in a weight ratio (acid:salt) within the range from about 0.2 to about 4.

In some embodiments, the acid may be created in situ from inclusion of the salt in an initial formulation along with an acid such as phosphoric acid or the like. In such an embodiment, the salt (e.g., sodium benzoate) interacts with the acid to form the aromatic acid in the composition. In other embodiments, the acid may be included in the initial formulation with a base such as sodium hydroxide, for example. Interaction between the aromatic acid and the base react to form the salt of the acid in situ.

Another component of the inventive compositions is one or more solvents, typically those that are nontoxic or exhibit low toxicity. Any of a variety of such solvents can be used in the compositions. One criteria for selecting a solvent is the stability of the solvent against oxidation. The solvent serves to prevent the destabilization of the composition by preventing the aromatic acid component from precipitating or crystallizing out of the composition, especially if the composition is to be exposed to lower temperatures (e.g., 5° C.) during storage or shipment, or if higher concentrations of aromatic acid are needed (e.g., 5%). Suitable solvents for use in the compositions described herein include but are not limited to glycols, alcohols, aprotic amides, esters, polyethers and combinations of the foregoing, for example. Some specific examples of suitable solvents include propylene glycol, ethanol, n-propanol, isopropanol, hexylene glycol, polyethylene glycol, glycerol, phenoxyethanol, butylene glycol and combinations of the foregoing. Solvent may be present within the compositions of the invention at a concentration from about 1% by weight to about 40% by weight and typically from about 5% to about 35% by weight.

Still another component of the inventive composition is a surfactant or wetting agent, typically an anionic surfactant. However, organic compounds that function as the solvent in the composition may also satisfy the function of the wetting agent. Glycols such as propylene glycol are exemplary of such dual functionality. Other suitable wetting agents include nonionic surfactants and alcohols. Most typically, an anionic surfactant is used and such surfactants often provide a detergent function that aids in disinfecting surfaces to which the composition is applied, particularly when soil is present on the surface.

Anionic surfactants can improve the protein denaturing ability of the compositions and increase the efficacy of the composition against catalase producing organisms and certain viruses. Exemplary of such anionic surfactants are salts of: alkyl sulfates, alkyl arylsulfates, alkyl sulfosuccinates, dialkyl sulfosuccinates, alkyl lactates (e.g., sodium alkyl lactates), alkyl alkoxylated sulfates, and xylene sulfonates. A particularly useful anionic surfactant is the sodium salt of dioctyl sulfosuccinate (DOSS). DOSS is relatively nontoxic and it improves solvency. Another useful surfactant is sodium capryl sulfate. The combination of anionic surfactants such as DOSS and salts of aromatic acids are particularly good combinations for denaturing proteins and viruses. Compositions comprising sodium benzoate with DOSS are effective in denaturing enzymes such as catalase and enhance the activity of the hydrogen peroxide against catalase producing organisms such as S. Aureus. Overall, anionic surfactants contribute to the disinfecting ability of the compositions of the invention and provide detergency function in the presence of soil and organic loads.

Other surfactants may also be used in this invention either alone or in combination with anionic surfactants. Such other surfactants may include amine oxides, phenol ethoxylates, fatty acid amides, sorbitan esters, fatty alcohol ethoxylates and block copolymers of ethylene oxide and propylene oxide such as that known under the trade designation “Pluronics” manufactured by BASF. In general, anionic surfactants may be used in formulations that are designed for the high level disinfection of certain endoscopes, for example.

Nonionic surfactants can be used either alone or in combination with anionic surfactants such as when long term stability of the composition may be a concern. Nonionic surfactants may also be used alone (e.g., without anionic surfactant) in formulations comprising an acidulant and a salt of an aromatic acid such as, for example, the combination of phosphoric acid and sodium benzoate, mentioned above, which generally has good water solubility.

The compositions of the invention also include a carrier for the ready to use formulations. Although any of a variety of carriers may be useful, typically, the compositions of the invention include water as the carrier. As such, the compositions of the invention are formulated as ready to use compositions having the above described components in concentrations that fall mainly within the described concentration ranges. The balance of the formulation is then comprised of the carrier (e.g., water) in an amount typically from about 50% to about 99% by weight. Most typically, the carrier is treated in some way to remove contaminants, especially particulates, potentially interfering ions or other chemicals and the like. Filtration, distillation and/or deionization are typical treatments for the carrier in order to render it relatively free of contaminants, undesired materials and the like.

Optional additional components may be included in the compositions of the invention such as: antifoaming agents, foaming agents, corrosion inhibitors, peroxide stabilizing agents, hydrotropes, fragrances, and colorants. Suitable corrosion inhibitors include nitrates, azoles such as benzotriazole, and imidazoles. Tin compounds and pyrophosphates are examples of suitable peroxide stabilizers. These optional components may be included in the compositions of the invention at a concentration level in the ready to use compositions of up to about 10% by weight. Exact amounts of the individual optional components is within the ordinary skill of those working in the art. Other components known to those skilled in the art may also be included in the composition to alter or tailor the basic composition to a particular need.

In the compositions of the invention, the combination of an aromatic acid, hydrogen peroxide, and surfactant has shown an improved mycobactericidal activity and a faster kill rate against pathogenic organisms when compared with known hydrogen peroxide formulations. Such known formulations include those based on aliphatic or phosphoric acids combined with hydrogen peroxide or combinations of peracetic acid and hydrogen peroxide. Furthermore, the compositions of this invention are active at higher pH values and generally have a buffering capacity when the solution pH is close to the pKa of the aromatic acid. The aromatic acids used in this invention are also inherently more stable toward oxidation compared to aliphatic acids used in the prior art providing a further advantage for their use with hydrogen peroxide. However, it will be appreciated that the inclusion of the aromatic acid(s) in the compositions of the invention does not preclude the inclusion of other acids such as the aforementioned acidulants including, for example, phosphoric acid, sulfuric acid, caprylic, lauric acid, citric acid and combinations thereof.

Many known disinfectants are often used improperly both in an industrial and hospital setting. For example, they may be applied to a surface and prematurely removed prior to allowing for the necessary contact or disinfection time. As a result, some pathogens may be reduced in number and not completely eliminated while other, more difficult pathogens, may not be killed at all. As a result, the surface that has been treated with these disinfectants is not disinfected, and the opportunistic transmission of the pathogen may be facilitated. The compositions of the invention typically provide faster kill rates against Mycobacteria, fungi, and other bacteria compared to known hydrogen peroxide based formulations. Consequently, shorter exposure times are more appropriate for the compositions of the invention.

The contact time for the compositions of this invention when used for high level disinfection tends to be a function of the aromatic acid and peroxide concentrations. Typical contact times for destroying/killing 10⁶ Mycobacterium species is two minutes or less. In sterilization applications, the time required is substantially longer than that required to achieve high level disinfection. Without being bound to a particular theory, it is believed that the improved activity for the compositions herein is related to the ability of the aromatic acid to penetrate the fatty outer lipid layer of the Mycobacterium. This fatty lipid layer normally protects the cell from the action of chemicals interacting with the layer such as traditional aqueous disinfectants (e.g., 7% H₂O₂). The fat soluble nature of the aromatic acids used herein is believed to permit the penetration of the organic acid which is enhanced by the inclusion of anionic surfactants and/or wetting agents in the composition. At lower hydrogen peroxide concentrations (e.g., 5% or less), the peraromatic acid is generally not formed at appreciable levels and does not contribute to the antimicrobial activity of the composition.

The non-corrosive properties of disinfectant compositions according to the invention may be further enhanced by the addition of certain corrosion inhibitors. The non-corrosive properties may be especially important in applications where the composition is to be applied to any of a variety of metal surfaces such as brass, aluminum, anodized aluminum, carbon steel and the like. Benzotriazole has been shown to have a beneficial effect in the compositions of the invention. Disinfectant compositions formulated with benzotriazole and having a pH of 4.0 -4.4 have been known to demonstrate a delay of about 2 weeks before the onset of corrosion on a brass surface following continuous exposure to the disinfectant composition. Benzotriazole is not generally needed to protect aluminum or ferrous metals, but it is useful with copper and its alloys. The compositions generally will not degrade or discolor engineering thermoplastics, o-rings, elastomers, or common household plastics. In addition, the compositions are non-corrosive and mild to the skin.

The compositions of the invention have a broad spectrum of activity and are capable of accomplishing high level disinfection on any of a variety of surfaces. Exemplary surfaces include the surfaces of delicate medical instruments, devices such as, for example, endoscopes, food contact surfaces, surfaces within ventilation ducts, on cruise ships, in hospitals, under and within carpeting, and the like. The compositions may also be use as disinfectant cleaners and as skin antiseptics. The compositions of the invention can be used for disinfecting dental, medical, and veterinary equipment and devices as well as disinfecting inanimate surfaces such as floors, furniture, ceilings, door knobs, toilet seats, building vents, and surfaces of sinks. In addition, the compositions are useful for treating and disinfecting agricultural goods, produce, and raw materials. Furthermore, because the compositions of this invention contain lower levels of peroxide (<6%), they may be used for antimicrobial skin cleaners (e.g., hand cleaners), washes and scrubs, antiseptics, and for direct antimicrobial use or as an additive to laundry or dishwashing formulations. The compositions can be directly applied to the skin or bodily orifices for the treatment of bacterial, viral, and fungal diseases such as acne or otitis externa. The compositions of this invention are useful as a bleach or hypochlorite replacement for cleaning and disinfecting surfaces including color fast fabrics and contaminated textiles. The compositions are especially useful for destroying spore forming molds and fungi such as those known to be direct causes of “sick building syndrome”.

The preparation of the compositions of the invention may be accomplished by mixing the components together in a suitable vessel. First the aromatic acid, aromatic acid salts, solvents, and surfactant are mixed and then stirred until all solids are dissolved. This initial step is generally carried out at room temperature, but the addition of heat may facilitate dissolution of solutes. Other components that are soluble in the nonaqueous solvent may also be added at this time. Such additional components may include certain corrosion inhibitors with poor solubility such as tolyltriazole. After dissolution of solids, water may be added to form an aqueous emulsion or a clear microemulsion. Other substantially water soluble ingredients may be added to the emulsion or microemulsion such as concentrated hydrogen peroxide. The pH of the resulting composition may then be adjusted, if necessary.

If two or more parts are premixed prior to their combination, concentrated hydrogen peroxide is typically added to a second part comprising water, aromatic acid, solvent, and surfactant. Additionally, the compositions may be prepared by mixing together three components such as (1) water, (2) concentrated hydrogen peroxide, and (3) a premixed component comprising aromatic acid, aromatic acid salt, solvent, and surfactant. It will be appreciated that the compositions of the invention may be manufactured in process steps that differ from the foregoing steps or that may be arranged in a different order than the forgoing steps. Variations to the preparation of the compositions of the invention are also contemplated.

The present invention further comprises methods of disinfecting and decontaminating surfaces. The methods of the invention comprise applying the composition to a surface for a period of time to achieve the desired result (e.g., high level disinfection, etc.). The compositions of the invention may be applied directly to the surface as a liquid, a spray, an aerosol, vapor, or in the form of nebulized drops. Conventional and non-conventional methods may be used for application including but not limited to: roll coating, dipping, spraying, or rotational tumbling. Once applied to the surface, the compositions are left on the surface to allow a sufficient exposure time to the microorganisms on the surface. Typical exposure times are from one to five minutes and generally from 30 seconds ten minutes. Following the exposure time, the composition is removed from the surfaces by rinsing. Typically, rinses comprising filtered water, alcohol, or aqueous alcohol solutions are suitable for removing the inventive compositions. After the compositions have been rinsed from the surface, the surface is dried. Drying can be accomplished using forced air blown over the surface or by simply allowing the surface to dry by evaporation under ambient temperature and humidity. An alcohol rinse is normally used for ease of drying small channels, orifices or other small surface structures on a decontaminated surface, such as on a medical instrument or the like.

The compositions of the invention can be provide in any of a variety of formats for use as a disinfectant. For example, the composition can be loaded into an applicator such as a wipe or sponge to provide a preloaded article that can be packaged as a ready to use item. Likewise, the compositions can be used in a conventional spray bottle or packaged in plastic refill container. The compositions of this invention may be provided initially as a concentrate and later diluted at the point of use. For example, a concentrated composition containing 7.9% by weight hydrogen peroxide, sodium benzoate, phosphoric acid, and anionic surfactant could be provided and later diluted with a solvent at a weigh ratio of 10:1 or the like to provide a ready-to-use disinfecting solution with a final peroxide concentration of about 0.79%. Such a solution would be suitable for killing a wide range of microorganisms including bacteria as well as viruses.

In another configuration, the components of the composition of the invention could be packaged into a multi-part (e.g., two-part) system so that the components of the final product are later mixed at the time of use. For example, hydrogen peroxide could be placed within a first container. A smaller second container containing the solvent, aromatic acid, and surfactant could be attached to the first container. At the time of use, the contents of the second container could then be added to the contents of the larger first container (containing the hydrogen peroxide) and mixed to thereby provide a ready-to-use composition according to the present invention. Alternatively, the composition of the invention could first be provided in a dry powder soluble in water. Such powders would employ percarbonates, sodium benzoate, a water soluble organic acid such as citric acid, a powdered anionic surfactant such as sodium lauryl sulfate. The overall composition could be a mix of dry powders which might dissolve quickly in water to provide a disinfecting composition.

Features of the preferred embodiment of the invention are further described in the non-limiting Examples set forth herein. Unless otherwise indicated, all parts and percentages are by weight.

### TEST PROCEDURES

The following test procedures were used in the various examples of the invention.

Procedure I: Microbial Kill Rate Assay

Compositions were challenged with test cultures of Staphylococus aureus (commercially available as ATCC # 6538 from American Type Culture Collection, Rockville, Md.), Escherichia coli (ATCC # 25922), and Pseudomonas aeruginosa (ATCC # 15442).

Bacteria were grown in Tryptic Soy Broth (TSB) (commercially available from Difco, Detroit, Mich.) at 35° C. for 16+/−2 hrs. A 0.3 ml culture suspension was spread on the surface of Tryptic Soy Agar (TSA) plate that was incubated at 35° C. for 16+/−2 hrs.

Bacterial cells were harvested from the agar plate with a glass L-rod by adding 1-3 ml of TSB and were transferred to a test tube. The resulting cell suspension was called the working culture.

A 25 ml Erlenmeyer flask containing a magnetic stirring bar was filled with 19 ml of a composition made according to the invention as described in the Examples. The flask was placed in a temperature controlled water bath equipped with stirring capability. The magnetic stirrer was turned on and temperature of the composition was adjusted to 23° C.+/−2° C. One ml of soil (Bovine Calf Serum commercially available from Hyclone, Logan, Utah) was added to the flask in order to perform the kill rate experiment in the presence of 5% soil.

At the start of each exposure time, 0.1 ml of Staphylococus aureus, Escherichia coli, or Pseudomonas aeruginosa working culture was added to the composition with soil. The exposure times were 30 seconds, 90 seconds, 2 minutes, and 5 minutes. At the end of each exposure time, 1 ml of suspension was transferred to a test tube containing 9 ml neutralizer (Bacto D/E Neutralizing Broth available from Difco) with 0.01 ml catalase (commercially available from ICN Pharmaceuticals, Inc., Costa Mesa, Calif.) to stop inactivation of bacteria. After vortexing, the neutralized 10⁻¹ cell suspension was further diluted to 10⁻² and 10⁻³ by transferring 1 ml into 9 ml D/E dilution blanks. From each of the three dilutions, 0.1 ml volume was plated onto a TSA plate and spread with the L-rod. The plates were incubated at 37° C. for 24 hrs and colony-forming units (CFU) were counted. The procedure was repeated using three replicate samples of each formulation. The diluted bacterial suspensions were plated in duplicate.

Microbial kill rate was reported as a log₁₀ reduction which was determined by calculating the difference between the log₁₀ of the initial inoculum count and the log₁₀ of the inoculum count after exposure to the compositions of the inventive Examples and of the comparative examples for about 30-second (T₃₀ₛ), 90-second (T₉₀ₛ), 2-minute (T₂ₘ), and 5-minute (T₅ₘ) intervals at about 23° C. The two duplicate plates at the selected dilution level were averaged and the initial inoculum count was calculated using the following formula:

Initial Inoculum Count=T₀=Ave. CFU of 3 replicates×1/dilution level×0.005 Where the sample inoculums were diluted (0.1 ml in 20.1 ml organic matter plus the compositions, the initial inoculum were reduced by 0.1 ml/20.1 ml, which equals 0.005.

For the test plates of each organism at each time period, the CFU's on all the 10⁻² and 10⁻³ plates were counted. The dilution level that had counts between 25 and 250 was determined. The two duplicate plates at the selected dilution level were averaged and the test plate count at the given time was calculated using the following formula:
T₃₀ₛ, T₉₀ₛ, T₂ₘ and T₅ₘ=Ave. CFU of 3 replicates×1/dilution level
Where the average plate count of 3 replicates are at intervals corresponding to 30 seconds, 90 seconds, 2 minutes, and 5 minutes.

The log reduction was determined by taking the logarithm to the base 10 of T₀, T₃₀ₛ, T₉₀ₛ, T₂ₘ, and T₅ₘ and using the following formulas:
Log reduction at 30 seconds=log₁₀T₀−log₁₀T₃₀ₛ
Log reduction at 90 seconds=log₁₀T₀−log₁₀T₉₀ₛ
Log reduction at 2 minutes=log₁₀T₀−log₁₀T₂ₘ
Log reduction at 5 minutes=log₁₀T₁−log₁₀T₅ₘ
Procedure II: Quantitative Tuberculocidal Suspension

0.1 ml volume of Mycobacterium terrae (ATCC 15755) grown in Middlebrook 7H9 Broth (commercially available from Difco) with Middlebrook ADC Enrichment (commercially available from Difco) was transferred to a 250 ml cell culture flask with a canted neck and a cap with a 0.2 μm filter containing 50 ml of Middlebrook 7H9 Broth supplemented with Middlebrook ADC Enrichment. The culture was incubated up to 2-4 weeks until the culture reached population around 10⁻⁷ M. terrae cells/ml. On the same day that the examples were run, 6 ml of the culture was transferred into a tissue grinder and homogenized manually for 10 min. The uniformity of culture was checked using a microscope. The population of the working suspension was determined by diluting serially the bacterial solution in saline and plating onto the surface of Middlebrook 7H11 Agar supplemented with Middlebrook AODC Enrichment (commercially available from Difco). The plates were incubated up to four weeks at 37° C. and CFUs were counted.

A small Erlenmeyer flask containing a magnetic stirring bar was filled with 8.5 ml of the composition in Examples 3, 4, and 10. The flask was placed on the magnetic stirrer and the solution was mixed to assure uniformity of the solution. 0.5 ml of soil (Bovine Calf Serum commercially available from Hyclone) was added to perform the kill rate experiment in the presence of 5% soil.

At the start of each exposure time, 1 ml of cell working suspension was added to the mixing compositions with soil. The typical exposure time consisted of 3 times which were selected from the following time intervals: 1, 2, 3, 5, and 10 minutes. At the end of each exposure time, 1 ml of suspension was transferred to a test tube containing 9 ml D/E broth as a neutralizer with 0.01 ml catalase. After vortexing, the neutralized 10⁻¹ solution suspension was further diluted to 10⁻²-10⁻⁷ by transferring 1 ml into 9ml D/E dilution blanks. From each dilution, 0.1 ml volume was plated into TSA plate spread with the L-rod. In some cases the suspension was filtered trough a Millipore filter which was previously wetted with approximately 10 ml of saline. After the filtration of the neutralized bacterial suspension, the filter was rinsed with 50 ml of saline. The filter with bacteria was aseptically transferred onto Midlebrook7H11agar plates supplemented with Enrichment AODC nutrients. The plates were incubated in a plastic bag to prevent drying at 35° C. for 4 weeks and CFUs were counted. The test was performed with three replicate samples of each composition.

Mycobactericidal activity was reported as a log₁₀ reduction, which was determined by calculating the difference between the log₁₀ of the initial inoculum count and the log₁₀ of the inoculum count after exposure to the compositions or components of the composition for specified intervals of time. The calculations were described in the Microbial Kill Rate Assay.

Procedure III: Fungicidal Activity of Disinfectant

Spores of Trichophyton mentagrophytes (ATCC 9344) were grown as in Association of Official Agricultural Chemists (AOAC) Official Method 955.17. The compositions were prepared and exposed to the bacteria as in the Microbial Kill Rate Assay except that the growth media specified in the AOAC method was used. Data analysis was preformed as described in the Microbial Kill Rate Assay.

### Ingredients

The components used in formulating the compositions described in the various Examples are listed In Table 1. Unless otherwise indicated, the components used were of food or pharmaceutical grade.

TABLE 1
Components

| | | | Commercial |
|---|---|---|---|
| Component | Trade Designation | Function/identity | Source/Address |
| Sodium benzoate | | Salt of benzoic acid | Avocado Research |
| | | (99%) | Chemicals, Ltd/ |
| | | | Heysham, England |
| Benzoic acid | | Aromatic acid | EM Science/Cherry |
| | | | Hill, NJ |
| Salicylic acid | | Aromatic acid (99+%) | Sigma-Aldrich |
| | | | Chemical Co./St Louis, |
| | | | MO |
| Phosphoric acid | | Acidulant (85%) | JT Baker Co. |
| | | | (Phillipsburg, NJ) |
| Hydrogen peroxide | SUPER D Stabilized | Peroxide source, | FMC Corp./South |
| | Hydrogen Peroxide | oxidizing agent | Charleston, WV |
| | (35% solution) | | |
| Sodium dioctyl | GEMTEX SC-40 | Anionic surfactant | Finetex, Inc./Spencer, |
| sulfosuccinate | | (40%) | NC |
| Sodium dioctyl | AEROSOL OT | Anionic surfactant | Cytec Industries/West |
| sulfosuccinate | | (100%) | Paterson, NJ |
| Sodium hydroxide | | pH adjustment | Mallinkrodt/Paris, |
| | | | Kentucky |
| 1,2 Propanediol | | Solvent, wetting agent | Sigma-Aldrich |
| | | | Chemical Co. |
| Isopropanol | | Solvent | EM Science |
| Benzotriazole | COBRATEC 99P | Corrosion inhibitor | PMC Specialties |
| | | | Inc./Cincinnati, OH |
| Benzotriazole | COBRATEC 35G | Corrosion inhibitor - | PMC Specialities Inc. |
| | | (35% in propylene | |
| | | glycol) | |
| Tolyltriazole | COBRATEC TT100 | Corrosion inhibitor | PMC Specialties Inc. |
| Distilled water | | Base/carrier | Premium Waters |
| | | | Inc./Minneapolis, MN |
| Lauric acid | | Aliphatic acid/ | Proctor and Gamble |
| | | Acidulant | Chemicals/ |
| | | | Cincinnati, OH |
| Decanol | | Solvent | Proctor and Gamble |
| | | | Chemicals |
| Disodium EDTA | | Chelating agent | Sigma-Aldrich |
| | | | Chemical Co. |
| Polydimethylsiloxane | Antifoam C | Antifoaming agent - | Dow Corning/Midland, |
| | | (30%) food grade | Michigan |
| polyoxamer | Pluronics P65 | Nonionic surfactant | BASF |
| Sodium dodecyl | Biosoft D-40 | Anionic surfactant | Stepan Co./Northfield, |
| benzenesulfonate | | (40%) | IL |
| Propylene glycol | | USP grade Solvent, | |
| | | wetting agent | |
| Sodium laurel sulfate | | Anionic surfactant | Sigma-Aldrich |
| | | | Chemical Co. |

### EXAMPLES

### Example 1

A ready-to-use composition suitable as a general use disinfectant was formulated to provide improved bactericidal activity and kill rate. The composition of Example 1 is described in Table 2.

TABLE 2
Example 1

| | Concentration |
|---|---|
| Ingredient | (wt. %) |
| Propylene glycol | 4.08 |
| Benzoic acid | 0.16 |
| Sodium benzoate | 0.15 |
| Sodium dioctyl sulfosuccinate | 0.41 |
| (GEMTEX SC-40) | |
| Hydrogen peroxide | 3.01 |
| Water | 92.19 |

First, the surfactant (sodium dioctyl sulfosuccinate), propylene glycol, benzoic acid, and sodium benzoate were stirred in a glass vessel for 1 hour. The surfactant contained trace amounts of isopropanol as received from the supplier. Next the composition was diluted with distilled water (152.11 grams), stirred briefly, and hydrogen peroxide was added. The remainder of the water was added so that the final weight was about 300 grams with a pH of 4.0.

The efficacy of the composition was evaluated using the Microbial Kill Rate Assay described in the Test Protocols above. The results are shown in Table 3.

TABLE 3
Bacteria Kill Rate - Example 1

| | | Log reduction for pathogenic bacteria | | |
|---|---|---|---|---|
| | Exposure Time | Staphylococus | Escherichia | Pseudomonas |
| Example No. | (seconds) | aureus | coli | aeruginosa |
| 1a | 30 | ≧8.20 | 5.90 | ≧8.32 |
| 1b | 90 | ≧8.20 | 6.90 | ≧8.32 |

The test results in Table 3 show a minimum kill of more than 99.999 percent of the pathogenic bacteria in 30 seconds. The composition of Example 1 exhibited no noxious fumes, odor, or skin contact hazards. These results were surprising in light of the face that a 3 percent USP hydrogen peroxide solution is known to require more than 15 minutes to provide an eight-log reduction of Staphylococus aureus. (See, e.g., FIG. 9-1, page 170 of Disinfection, Sterilization, and Preservation edited by Seymour S. Block; 4^{th} Edition, 1991).

### Example 2

### Mycobactericidal Composition

A mycobactericidal composition was made using the components in the amounts given in Table 4. The surfactant, lauric acid, isopropanol, a corrosion inhibitor, and sodium benzoate were stirred in a glass vessel for 1 hour. Next, the composition was diluted with 51.62 grams of distilled water, stirred briefly, and hydrogen peroxide and phosphoric acid were added. Finally the remainder of the water was added. The final pH was 4.2. This composition uses sodium benzoate in conjunction with phosphoric acid and lauric acid as acidulants. The phosphoric acid is believed to interact with the sodium benzoate to provide benzoic acid in the resulting composition.

TABLE 4
Example 2

| | Mass | Concentration |
|---|---|---|
| Components | (g) | (wt. %) |
| Lauric acid | 0.26 | 0.26 |
| Isopropanol | 5.21 | 5.20 |
| Sodium dioctyl sulfosuccinate | 4.55 | 1.82 |
| (GEMTEX SC-40) | | |
| Benzotriazole (COBRATEC 99P) | 2.54 | 2.54 |
| Sodium benzoate | 1.04 | 1.04 |
| Phosphoric acid | 9.10 | 0.46 |
| Stabilized hydrogen peroxide | 14.48 | 5.06 |
| Propylene glycol | 4.81 | 4.80 |
| Distilled water | 58.16 | 78.82 |

The composition was tested using Procedure II (Mycobactericidal Activity Of Disinfectant). The results demonstrated >7.6 log reduction of mycobacteria after an exposure time of 2 minutes in the presence of 5 percent fetal bovine serum.

### Example 3

### Mycobactericidal Composition

A composition was prepared using the components in the amounts given in Table 5. The first six components were added together and were stirred in a glass vessel at room temperature for 30 minutes. Thereafter, 48.50 grams of distilled water was added followed by each additional component as listed. The components were added individually followed by stirring to dissolution. The composition had a pH of 4.5. Sodium salicylate, the salt of salicylic acid, was expected to be formed from the combination of salicylic acid and sodium hydroxide.

TABLE 5
Example 3

| | Mass | Concentration |
|---|---|---|
| Components | (g) | (wt. %) |
| Benzotriazole (COBRATEC 99P) | 7.09 | 6.47 |
| Propylene glycol | 2.50 | 2.28 |
| Sodium dioctyl sulfosuccinate | 1.02 | 0.37 |
| (GEMTEX SC-40) | | |
| Benzoic Acid | 0.47 | 0.43 |
| Isopropanol | 4.01 | 3.66 |
| Sodium benzoate | 0.62 | 0.57 |
| Phosphoric acid | 9.10 | 0.45 |
| Hydrogen peroxide | 14.52 | 4.63 |
| Distilled water | 70.26 | 81.13 |
| Antifoam C | 0.06 | 0.01 |

The composition was tested using Procedure II (Quantitative Tuberculocidal Suspension) for activity against Mycobacterium terrae in the presence of 5% bovine serum. This composition showed ≧5.9 log reduction of Mycobacterium terrae at 2 minute exposure times.

The concentration of actives in this test based on dilution with Mycobacterium terrae and calf serum was 4.32% hydrogen peroxide and 0.93% benzoic acid/sodium benzoate (total benzoic acid content). These results indicate that a composition with a high-level disinfection time of 2 minutes is achievable. The composition was nonirritating to the skin.

### Example 4

### Mycobactericidal Composition

A composition was made with salicylic acid and the other components in the amounts given in Table 6. The first four components were mixed with stirring followed by the addition of 51.15 grams of distilled water. The remainder of the components were added in the order listed followed by stirring and with more water added as the last component to provide the concentrations shown in the Table. The composition used a higher alcohol and surfactant concentration because of the poor solubility of tolyltriazole (corrosion inhibitor for copper alloys). The final concentration of salicylic acid was 1.02% and hydrogen peroxide was 4.22%.

TABLE 6
Example 4

| | Mass | Concentration |
|---|---|---|
| Components | (g) | (wt. %) |
| Isopropanol | 9.00 | 9.00 |
| Sodium dioctyl sulfosuccinate | 10.48 | 4.19 |
| (GEMTEX SC-40) | | |
| Salicylic acid | 1.02 | 1.02 |
| (2-hydroxybenzoic acid) | | |
| Tolyltriazole | 2.04 | 2.04 |
| Hydrogen peroxide | 12.06 | 4.22 |
| Sodium hydroxide | 0.66 | 0.04 |
| Distilled water | 64.74 | 79.49 |

The composition was tested using Procedure II (Quantitative Tuberculocidal Suspension) for activity against Mycobacterium terrae in the presence of 5% bovine serum with an exposure time of 5 minutes. In a kill rate assay, the composition provided a complete kill (≧7.69 log reduction) against Mycobacterium terrae.

### Comparative Example A and Examples 5-8

To examine the ability of compositions of the invention to significantly reduce the activity of catalase by denaturing it, catalase activity was measured in a foaming experiment. Catalase is a known catalytic enzyme which decomposes hydrogen peroxide into oxygen and water. Consequently, bacteria containing a large amount of catalase (e.g., S. Aureus) have a built in defense mechanism against hydrogen peroxide disinfectants. Comparative Example A was a 3% hydrogen peroxide solution (no additives. Examples 5-8 were formulated as set forth in Table 7.

1 ml of bovine calf serum was added to 19 ml of hydrogen peroxide disinfectant composition in a graduated cylinder while stirring with a magnetic stir bar. The solution was stirred slowly on a stir bar plate. 0.1 ml of active bovine liver catalase was added to each composition and the catalase reacted with the hydrogen peroxide to generate oxygen, causing the protein to foam. After two minutes, the volume of foam was recorded. The results are shown in Table 8.

TABLE 7
Components (Comparative Example A and Examples 5-8)

| | Components (weight percent) | | | | | | | |
|---|---|---|---|---|---|---|---|---|
| | | | | | | | Sodium dioctyl | |
| Example | Hydrogen | Propylene | Benzoic | Sodium | Silicone | | sulfosuccinate | |
| Number | peroxide | glycol | acid | benzoate | antifoam | Ethanol | (GEMTEX SC-40) | Water |
| Comp. Ex. A | 3.00 | 0.00 | 0.00 | 0.00 | 0.00 | 0.00 | 0.00 | 97.00 |
| 5 | 5.10 | 6.80 | 0.31 | 0.35 | 0.00 | 0.00 | 0.00 | 87.44 |
| 6 | 5.10 | 6.10 | 0.25 | 0.25 | 0.02 | 0.00 | 0.00 | 88.28 |
| 7 | 5.00 | 6.20 | 0.26 | 0.27 | 0.01 | 3.50 | 1.50 | 83.26 |
| 8 | 5.00 | 6.20 | 0.25 | 0.25 | 0.00 | 6.90 | 3.22 | 78.18 |

TABLE 8
Foam Volume (Comparative Example A and Examples 5-8)

| Example | Foam Volume (ml) |
|---|---|
| Comp. Ex. A | 100 |
| 5 | 10 |
| 6 | 10 |
| 7 | 0 |
| 8 | 1 |

Similar results were obtained when the bovine catalase was replaced with a 10⁸/mL innoculum of Staphylococus aureus, which was grown in an open plate to maximize catalase content.

### Example 9

A composition was prepared with 0.45% benzoic acid, 0.50% sodium benzoate, 2.30% benzotriazole, 0.48% sodium dioctyl sulfosuccinate, 5.03% hydrogen peroxide, 5.87% propylene glycol, and 3.84% isopropanol. The composition was tested against Staphylococcus aureus (ATCC 6538), an organism containing a large amount of catalase, using Procedure I (Microbial Kill Rate Assay) with 5% calf serum. The composition provided complete kill at 2 minutes with ≧6.82 log reduction.

### Comparative Example B

A composition was prepared with the components listed and in the amounts given in Table 9. The first five components were added together and stirred in a glass vessel at room temperature for 30 minutes. Thereafter, each additional component was added individually followed by stirring to dissolution. The composition of Comparative Example B was formulated with lauric acid, a C₁₂ aliphatic acid, and included no aromatic acid component and no salt of an aromatic acid.

TABLE 9
Components and amounts for Comparative Example B

| | Mass | Concentration |
|---|---|---|
| Components | (g) | (wt. %) |
| Sodium dioctyl sulfosuccinate | 6.42 | 2.56 |
| (GEMTEX SC40) | | |
| Lauric acid | 1.03 | 1.02 |
| polydimethylsiloxane | 0.21 | 0.01 |
| (Antifoam C) | | |
| Decanol | 1.01 | 1.01 |
| polyoxamer | 4.17 | 4.16 |
| Distilled water | 63.10 | 81.06 |
| Isopropanol | 5.11 | 5.10 |
| Disodium EDTA | 5.01 | 0.12 |
| Hydrogen peroxide | 14.20 | 4.96 |

The composition was tested in triplicate according to Procedure II (Quantitative Tuberculocidal Suspension), resulting in a <3.3 log reduction in Mycobacterium terrae after a 5 minute exposure time. The results indicated that the composition of Comparative Example B was not mycobactericidal in 5 minutes, in contrast to the compositions of the inventive examples.

### Example 10

### Mycobactericidal Composition with Antifoam and Corrosion Inhibitor

A composition was made using the components in the amounts given in Table 10. The first seven components were stirred in a glass vessel for 1 hour. Next the composition was diluted with 50.33 grams distilled water, stirred briefly, and hydrogen peroxide and (Antifoam C) were added. Finally the remainder of the water was added to provide the concentrations shown in the table. The composition had a pH of 4.3.

TABLE 10
Example 10

| | Mass | Concentration |
|---|---|---|
| Components | (g) | (wt. %) |
| Isopropanol | 5.02 | 5.02 |
| Propylene glycol | 2.01 | 2.01 |
| Benzotriazole | 2.00 | 2.00 |
| (COBRATEC 99P) | | |
| 1,2-propanediol | 3.00 | 3.00 |
| Benzoic acid | 0.63 | 0.63 |
| Sodium benzoate | 0.58 | 0.58 |
| Sodium laurel sulfate | 0.31 | 0.31 |
| polydimethylsiloxane | 0.20 | 0.04 |
| (Antifoam C) | | |
| Hydrogen peroxide | 14.75 | 5.17 |
| Distilled water | 71.44 | 81.24 |

This composition showed a 3.7 log reduction of Mycobacterium terrae at 1 minute and a 5.5 log reduction after 2 minute exposure times in the Quantitative Tuberculocidal Suspension Method described in the Test Protocols with 5% calf serum.

### Example 11

### Mycobactericidal Composition with Containing Soil and Hard Water

300 ml of a composition was prepared using the components in the amounts given in Table 11. The first seven components were stirred in a glass vessel for 1 hour. Next, the composition was diluted with 148 ml of distilled water while stirring on a magnetic stirring plate, and hydrogen peroxide was then added, followed by addition of the remainder of the water. The composition had a pH of 4.2.

TABLE 11
Example 11

| | | Concentration |
|---|---|---|
| | Mass | (wt. %) |
| Components | (g) | prepared |
| Isopropanol | 27.06 | 9.02 |
| Propylene glycol | 44.64 | 14.88 |
| Benzoic acid | 3.05 | 1.02 |
| Sodium benzoate | 1.51 | 0.50 |
| Sodium laurel sulfate | 1.12 | 0.37 |
| Hydrogen peroxide | 45.00 | 15.00 |
| Distilled water | 177.63 | 59.21 |

Hard water was prepared per the AOAC definition of hard water described in AOAC Official Method 955.17. Thereafter, 40 ml of the composition was diluted by adding 10 ml of synthetic hard water to obtain 80% concentration of the original composition in hard water. The effect of hard water and soil on the ability of the composition to kill Mycobacterium terrae was then investigated by adding to each of several reaction flasks 9 ml of the diluted composition, 1 ml of Mycobacterium terrae suspension, containing 5% bovine calf serum. The diluted composition of Example 11 showed a 3.2 log reduction of Mycobacterium terrae at 1 minute and ≧7.01 log reduction after 2 minute exposure times following Procedure II—Quantitative Tuberculocidal Suspension Assay with 5% calf serum.

Various embodiments of the invention have been described as foreseen by the inventor for which an enabling description was available. It should be appreciated that insubstantial modifications of the invention, not presently foreseeable by those of reasonable skill in the art, may nonetheless represent equivalents thereto.

1. A composition, comprising:
Greater than about 0.1% by weight hydrogen peroxide;
Aromatic acid component;
Surfactant;
Optionally, a solvent; and
A carrier.

2. Composition of claim 1 formulated to provide a pathogenic bacteria kill rate of 99.9% in about 90 seconds when bacteria are exposed to the composition.

3. Composition of claim 1 wherein the aromatic acid component comprises an aromatic acid and a salt of the aromatic acid.

4. Composition of claim 1 further comprising an acidulant selected from the group consisting of phosphoric acid, sulfuric acid, caprylic acid, capric acid, lauric acid, or citric acid and combinations of the foregoing.

5. Composition of claim 1 wherein the hydrogen peroxide is present in the composition at a concentration from about 0.1% to about 7% by weight.

6. Composition of claim 1 wherein the aromatic acid component is present in a concentration sufficient to provide a disinfecting action when used to kill microorganisms such as bacteria, fungi and viruses.

7. Composition of claim 1 wherein the aromatic acid component is present in a concentration sufficient to provide a synergy when combined in the composition with the hydrogen peroxide as a disinfecting composition to kill microorganisms such as bacteria, fungi and viruses.

8. Composition of claim 1 wherein the aromatic acid component is selected from the group consisting of benzoic acid, alkyl derivatives of benzoic acid, hydroxybenzoic acids, halogenated benzoic acids, phthalic acid, terephthalic acid, orthophthalic acid, acetylsalicylic acid, napthoic acid and combinations of the foregoing, the aromatic acid component present in a concentration from 0.1% by weight to 5% by weight.

9. Composition of claim 1 wherein the surfactant is anionic.

10. Composition of claim 9 wherein the surfactant is selected from the group consisting of alkyl sulfates, alkyl arylsulfates, alkyl sulfosuccinates, dialkyl sulfosuccinates, and xylene sulfonates, salts thereof and combinations of the foregoing.

11. Composition of claim 9 wherein the surfactant is the sodium salt of dioctyl sulfosuccinate.

12. Composition of claim 1 wherein the surfactant is selected from the group consisting of amine oxides, phenol ethoxylates, fatty acid amides, sorbitan esters, fatty alcohol ethoxylates, block copolymers of ethylene oxide and propylene oxide and combinations of the foregoing.

13. Composition of claim 1 wherein the solvent is selected from the group consisting of glycols, alcohols, aprotic amides, esters, polyethers and combinations of the foregoing.

14. Composition of claim 1 wherein the carrier is water and the composition is an emulsion.

15. Composition of claim 1 further comprising one or more optional constituents selected from corrosion inhibitors, antifoaming agents, foaming agents, pH adjusting agents, coloring agents, peroxide stabilizing agents, fragrances, and chelating agents.

16. Composition of claim 1 having a pH in the range from about 3.5 to about 5.0.

17. Composition of claim 1 wherein:
The concentration of hydrogen peroxide is from about 1% by weight to about 7% by weight; and
The concentration of aromatic acid component is from about 0.1% by weight to about 5% by weight.

18. Composition of claim 17 further comprising a salt of the aromatic acid in a concentration less than about 2% by weight.

19. Composition of claim 18 wherein the aromatic acid is benzoic acid and the salt is sodium benzoate.

20. Composition of claim 19 wherein the surfactant comprises salts of compounds selected from the group consisting of alkyl sulfates, alkyl arylsulfates, alkyl sulfosuccinates, dialkyl sulfosuccinates, alkyl lactates, alkyl alkoxylated sulfates, xylene sulfonates and combinations of the foregoing.

21. Composition of claim 19 wherein the surfactant is selected from the group consisting of the sodium salt of dioctyl sulfosuccinate, sodium capryl sulfate and combinations of the foregoing.

22. Composition of claim 12 further comprising an antifoaming agent.

23. Composition of claim 22 further comprising acidulant selected from the group consisting of phosphoric acid, sulfuric acid, caprylic acid, capric acid, lauric acid, citric acid and combinations of the foregoing.

24. Composition of claim 17 wherein the solvent is selected from the group consisting of propylene glycol, ethanol, n-propanol, isopropanol, hexylene glycol, polyethylene glycol, glycerol, phenoxyethanol, butylene glycol and combinations of the foregoing.

25. Composition of claim 24 wherein the solvent concentration is from about 1% to about 40% by weight.

26. Composition of claim 17 wherein the composition is more resistant to catalase deactivation than an aqueous solution of hydrogen peroxide.

27. Composition of claim 17 having a pH in the range from about 3.5 to about 5.0.

28. A method for disinfecting, the method comprising:
Applying the composition of claim 1 to a substrate;
Allowing the composition to remain in contact with the substrate for a period of time to kill microorganisms thereon; and
Removing the composition from the substrate.

29. Method according to claim 28 wherein the substrate is a surface on a medical instrument.

30. Method according to claim 28 wherein the medical instrument is an endoscope.

31. Method according to claim 28 wherein applying the composition of claim 1 to the substrate is accomplished by roll coating, dipping, spraying, or rotational tumbling.

32. Method according to claim 28 wherein the period of time ranges from 30 seconds to ten minutes.

33. Method according to claim 28 wherein the period of time is no longer than about two minutes and the microorganisms comprise Mycobacterium, the method providing a kill of 10⁶ Mycobacteria within the period of time.

34. Method according to claim 28 wherein removing the composition from the substrate is accomplished by rinsing with water, alcohol, or aqueous alcohol solution.

35. Method according to claim 28 further comprising drying the substrate after removing the composition

36. Method according to claim 28 where the substrate is a hard surface or a textile.

37. A method for making a composition, the method comprising combining the initial components of claim 1 to provide the composition.

38. Composition according to claim 1 formulated for application to skin.
